(19) United States
(12) Patent Application Publication
(10) Pub. No.: US 2025/0099367 A1
(43) Pub. Date: Mar. 27, 2025

(54) TARGETED DELIVERY OF MODIFIED RNA

(71) Applicant: The Chinese University of Hong Kong, Shatin (CN)

(72) Inventors: Oi Lan Kathy LUI, Shatin (CN); Hung Fat TSE, Hong Kong SAR (CN); Kwong-Man NG, Hong Kong SAR (CN); Jennifer ZHANG, Hong Kong SAR (CN)

(21) Appl. No.: 18/890,660

(22) Filed: Sep. 19, 2024

Related U.S. Application Data
(60) Provisional application No. 63/584,188, filed on Sep. 21, 2023.

(51) Int. Cl. A61K 9/00; A61K 38/47; A61K 48/00

(52) U.S. Cl. CPC A61K 9/0009; A61K 38/47; A61K 48/005; A61K 48/0075; C12Y 302/01022

(57) ABSTRACT

The present invention provides methods involving the use of microbubbles and ultrasound for targeted delivery of a modified RNA encoding a protein of interest as a means for effective transfer of genetic material for various therapeutic purposes. Corresponding compositions and kits useful for practicing such methods are also provided.

### RELATED APPLICATIONS

This application claims priority to U.S. Provisional Patent Application No. 63/584,188, filed Sep. 21, 2023, the contents of which are hereby incorporated by reference in the entirety for all purposes.

### BACKGROUND OF THE INVENTION

Nucleic acid-based therapy or gene therapy is a drug delivery research field that has attracted increased attention since the 1980s. The exogenous nucleic acids upon being delivered into the cells of a targeted tissue or organ are either translated into proteins, interfere with target gene expression, or possibly correct genetic mutations. The most common form of gene therapy uses a nucleic acid encoding a functional protein to replace or supplement a lost or inactivated protein due to a mutated gene carried by a patient, thus achieving the desired therapeutic benefits. Frequently, gene therapy focuses on diseases caused by single-gene defects, such as cystic fibrosis, hemophilia, muscular dystrophy, thalassemia, Parkinson's disease, sickle cell anemia, familial hypercholesterolemia, and hereditary hemochromatosis. In addition, various types of cancer have been the subject matter of gene therapy testing, including blood malignancies (e.g., leukemia and lymphoma) as well as solid tumors (e.g., breast, colon, liver, stomach cancer and melanoma). Despite intense research efforts, therapeutic strategies involving nucleic acid transfer continue to suffer from short-comings such as limited target specificity and delivery efficiency, which in turn can lead to adverse side-effects. As such, there exists an urgent need for developing new and effective methods useful in nucleic acid delivery for therapeutic purposes. This invention fulfills this and other related needs.

### BRIEF SUMMARY OF THE INVENTION

The present invention relates to a highly effective and target-specific gene delivery method utilizing microbubbles to carry a modified ribonucleic acid (RNA) and ultrasound to release the modified RNA to a target cell, for example, located at a pre-selected anatomic site within a recipient's body. Thus, in a first aspect, this invention provides a method for delivering to a target cell a nucleic acid, especially a modified ribonucleic acid (RNA), which encodes a protein of interest. The method includes these steps: (i) contacting the cell a composition comprising microbubbles and the nucleic acid encoding the protein of interest, and (ii) applying ultrasound to the target cell, thereby introducing the nucleic acid into the target cell.

In some embodiments, the method further includes, following step (ii), a step of maintaining the target cell under permissible conditions so as to allow the protein of interest to be expressed in the target cell. In some embodiments, the target cell is present at an anatomic site within a patient's body, and step (i) comprises administering to the patient an effective amount of the composition, and step (ii) comprises applying ultrasound to the anatomic site. In some embodiments, where the target cell is located within a patient's body, step (i) comprises injection of the composition to the patient, for instance, by intravenous, intramuscular, subcutaneous, intraperitoneal, or intratumoral injection. In some cases, the patient suffers from a disease characterized by a deficiency of the protein of interest, such as cancer, Fabry disease (FD), gastrointestinal intolerance of oligosaccharides, Parkinson's disease, familial hypercholesterolemia, and hereditary hemochromatosis. The patient may be a human or a non-human mammal, especially another primate. In some embodiments, the disease to be treated is Fabry Disease (FD) and the protein of interest is α-galactosidase (αGAL). In some embodiments, the target cell is located at the anatomic site of heart, liver, or kidney. In some embodiments, the modified RNA encodes αGAL and comprises the polynucleotide sequence set forth in SEQ ID NO:1. In some embodiments, the modified RNA comprises 5-methylcytidine and pseudouridine, replacing the cytidine and uridine residues, respectively. In some embodiments, the microbubbles are about 1 μm to about 11 μm in diameter, e.g., about 5 μm or less than about 8 μm on average. In some embodiments, the microbubbles comprise a phospholipid and sulphur hexafluoride. In some embodiments, the modified RNA is administered to the patient by intravenous injection at a dose of about 0.5 mg/kg per injection. In some embodiments, the modified RNA is administered to the patient at a frequency of no more than once about every 42 days.

In a second aspect, the present invention provides a composition for RNA delivery and potentially for treating a disease, comprising (1) an effective amount of a modified RNA encoding a protein of interest; (2) microbubbles; and (3) a physiologically acceptable excipient. In some embodiments, the composition is formulated for administration by injection, e.g., intravenous, intramuscular, subcutaneous, intraperitoneal, or intratumoral injection. In some embodiments, the modified RNA an αGAL and comprises the polynucleotide sequence set forth in SEQ ID NO:1. In some embodiments, the modified RNA comprises 5-methylcytidine and pseudouridine. in some embodiments, the composition may further comprise a therapeutic agent known to be effective for treating a disease the protein of interest is intended to cure (e.g., an anti-cellular proliferation agent for treating a cancer or a drug for treating FD). In some embodiments, the microbubbles are about 1 μm to about 11 μm in diameter, e.g., having an average diameter of about 5 μm or less than 8 μm. In some embodiments, the microbubbles comprise a phospholipid and sulphur hexafluoride.

In a third aspect, the present invention provides a kit for treating a disease characterized by a deficiency of a protein of interest. The kit comprises; (1) a first container containing a first composition comprising microbubbles and an effective amount of a modified RNA encoding the protein of interest; and (2) a second container containing a second composition comprising an effective amount of another therapeutic agent for the disease. For example, if the disease being treated is a disease involving excessive or inappropriate cellular proliferation, the second composition may comprise an anti-proliferation agent (e.g., anti-cancer drug). In the case of a kit for treating FD, the second composition may comprise a drug known to be effective for treating FD. In some embodiments, the first composition is formulated for administration by injection, e.g., intravenous, intramuscular, subcutaneous, intraperitoneal, or intratumoral injection. In some embodiments, the modified RNA encodes an αGAL and comprises the polynucleotide sequence set forth in SEQ ID NO:1. In some embodiments, the modified RNA comprises at least one possibly more substitution with non-naturally occurring nucleotide or nucleoside such as 5-methylcytidine and pseudouridine. In some embodiments, the microbubbles are about 1 μm to about 11 μm in diameter, for example, having an average diameter of about 5 μm or less than 8 μm. In some embodiments, the microbubbles comprise a phospholipid and sulphur hexafluoride. In some embodiments, the kit may further comprise an instruction manual for administration of the first and second compositions.

### BRIEF DESCRIPTION OF THE DRAWINGS

FIG. 1: Transfection of GLA-modRNA restores αGAL protein production and enzymatic activity in cardiomyocytes derived from Fabry disease patient-specific iPSCs. 400 ng GFP-modRNA or GL4-modRNA were transfected into wildtype and GLA-deficient iPSC-CMs, respectively, for analysis. (A) The culture media were collected at 3 days after transfection. The relative levels of αGAL proteins in the spent media were evaluated by Western Blot. (B) Quantification of (A) (C) αGAL activities of the spent media collected at 3 days after transfection. Data are represented as mean+/−SEM. from 3 independent biological repeats; *P<0.05, **P<0.01.

FIG. 2: Ultrasound mediated sonoporation enhances targeted tissue delivery of GLA-modRNA in GLA-deficient mice. 120 μl of saline GLA-modRNAs (0.5 μg/g) were injected intravenously into GLA-KO mice. Mice were sacrificed at 3 days after injection. The plasma (A), heart (B), liver (C) and kidneys (D) were harvested for the evaluation of αGAL-enzyme activities. Data are represented as mean+/−SEM. from 3 mice per group; *P<0.05.

FIG. 3: Ultrasound mediated sonoporation enhanced targeted tissue delivery GLA-modRNA in-vivo. The GLA-deficient mice (n=6) received either tail vein injection of GLA-modRNA (0.5 mg/kg) with ultrasound over the chest, or tail vein injection of αGAL protein (αGAL-ERT). Plasma was collected at regular time intervals as indicated, and mice were sacrificed at 42 days after injection. (A) Plasma and (B) heart tissue lysates were subjected to αGAL-enzyme activity evaluation. The enzymatic activity levels are compared with that of wildtype control group. (C) The change in plasma Lyso-Gb3 levels. Data are represented as mean+/−SEM from 6 animals of each group. *P<0.05: **P<0.01.

FIG. 4: Targeted tissue delivery of microbubble encapsulated GLA modRNA restores αGAL activity and function in mouse model of FD. Immunohistochemistry analysis of the αGAL protein distributions was carried out in the livers, hearts and kidneys. Wildtype and GLA-KO mice receiving GLA-modRNA or αGAL-ERT were evaluated with antibodies specific to αGAL, visualised using DAB substrate kits and counter stained with haematoxylin. Scale bars=100 um.

FIG. 5: Lack of immunogenicity against the application of microbubble coated GLA-modRNA in vivo. Microbubble-mediated GI-modRNA delivery did not elicit immune response. (A) Liver function test was determined by measuring plasma levels of alanine transaminase (ALT) and aspartate transaminase (AST). (B) Relative plasma anti-αGAL-antibody titers as determined by ELISA assay. (C) Quantification of flow cytometry results showing the percentage of CD4+ and CD8+ T cells in the spleen. (D) Quantification of flow cytometry results showing the M1/M2 macrophage ratio. (E) Relative plasma levels of pro-inflammatory and anti-inflammatory cytokine gene expression as determined by LEGENDplex™ Mouse Inflammation Panel.

FIG. 6. Characterization of iPSC-CMs derived from patients with Fabry Disease. (A) The iPSCs lines were generated from 2 patients with Fabry diseases (FD1 and FD2) carrying the same GLA mutation (IVS4+ 919G→A mutation). The expression of OCT4 and SSEA4 proteins (pluripotency markers) were confirmed by immunofluorescent staining. Scale bar=100 μm. (B) The iPSCs lines were differentiated into cardiomyocytes (iPSC-CMs) and the expression of cardiac troponin-T (cTnT) and αGAL were evaluated by immunofluorescent staining. Scale bar=50 μm.

FIG. 7. Localization of modRNA encapsulated by SonoVue after systemic administration. 50 ug GFP-modRNA loaded on microbubble (Sonovue) was injected into each mouse via tail vein in the presence/absence of ultrasound over the chest. At day 1 after intravenous injection, the mouse was sacrificed and the eGFP levels in different vascularized tissues including the heart, liver, spleen and kidney were evaluated using in vivo imaging system (IVIS).

### DEFINITIONS

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

A “modified nucleic acid” encompasses both DNA and RNA molecules that contain one or more nucleotide residues where one or more non-naturally occurring nucleotides have replaced the corresponding naturally occurring nucleotides while retaining the same Watson-Crick base-pairing capability so as to ensure the same protein product is encoded by the DNA or RNA molecule before and after it is modified.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, the term “microbubble” refers to a micrometer-range bubble formed by a biocompatible material (e.g., macromolecules such as lipids, proteins, or polymers such as surfactants) shell encapsulating a gas core. The shell may include one or more types of molecules, which lower the interfacial tension between the gas core and the exterior aqueous environment, such as a physiological environment. The shell may comprise, for example, lipids (e.g., phospholipids), proteins (e.g., albumin), sugars, and/or polymers. In general, the microbubbles have an average diameter of no more than about 10 μm, although they can encompass bubbles less than 1 μm (i.e., nanobubbles) in diameter, for example, about 100 nm-1 μm, about 200 nm-1 μm, or about 300 nm-1 μm. In some cases, the average microbubble size within a microbubble composition is at least about 1, 2, 3, 4, or 5 μm. For example, the average microbubble size is about 1-10, 1-5, 1-4, 1-3, 1-2, 2-5, 2-4, 2-3, 3-5, or 3-4 μm.

The term “inhibiting” or “inhibition,” as used herein, refers to any detectable negative effect on a target biological process, such as protein phosphorylation, cellular signal transduction, protein synthesis, cell proliferation, tumorigenicity, and metastatic potential etc. Typically, an inhibition is reflected in a decrease of at least 10%, 20%, 30%, 40%, or 50% in the target process (e.g., target cell proliferation, or target gene expression), or any one of the downstream parameters mentioned above, when compared to a control. In a similar fashion, the term “increasing” or “increase” is used to describe any detectable positive effect on a target biological process, such as a positive change of at least 25%, 50%, 75%, 100%, or as high as 2, 3, 4, 5 or up to 10 or 20 fold, when compared to a control. In contrast, the term “substantially the same” or “substantial lack of change” indicates little to no change in quantity, typically within ±10% of a control value, or within ±5%, 2%, or even less variation from the control value.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context does not require 100% elimination of the occurrence of an event, rather, it refers to a suppression or reduction in the likelihood or severity of such occurrence or a delay in such occurrence.

A “subject,” or “subject in need of treatment,” as used herein, refers to an individual who seeks medical attention due to risk of, or actual sufferance from, a condition involving, caused by, or exacerbated by a deficiency of the expression of a protein of interest. The term subject can include both animals, especially mammals, and humans. Subjects or individuals in need of treatment include those that demonstrate symptoms of undesirable or inappropriate cell proliferation such as tumor and especially malignant tumor/cancer or are at risk of later developing these conditions and/or related symptoms.

The term “cancer” encompasses any of various malignant neoplasms characterized by the proliferation of anaplastic cells that tend to invade surrounding tissue and metastasize to new body sites. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma. Hodgkin's lymphoma, B-cell lymphoma, or Burkitt's lymphoma), and multiple myeloma.

The term “effective amount,” as used herein, refers to an amount that is sufficient to produces an intended effect for which a substance is administered. The effect may include a desirable change in a biological process (e.g., increased expression of a protein of interest, decreased cellular proliferation, or decreased inflammation) as well as the prevention, correction, or inhibition of progression of the symptoms of a disease/condition and related complications to any detectable extent. The exact amount “effective” for achieving a desired effect will depend on the nature of the therapeutic agent, the manner of administration, and the purpose of the treatment, and will be ascertainable by one skilled in the art using known techniques (see, e.g., Lieberman, Pharmaceutical Dosage forms (vols. 1-3, 1992); Lloyd, The Art, Science and Technology of Pharmaceutical Compounding (1999); and Pickar. Dosage Calculations (1999)).

A composition “consisting essentially of” an active ingredient is a composition that includes the active ingredient having a predetermined and desired biological activity or efficacy (for example, the expression of a protein of interest or cellular proliferation rate) but no other compounds that contribute to or affect in any detectable manner the predetermined biological activity or efficacy of the active ingredient. Such a composition may include inactive excipients, e.g., for formulation or stability of a pharmaceutical composition, and/or additional active ingredients that have unrelated activity but do not detectably contribute to the predetermined biological activity or efficacy.

A “pharmaceutically acceptable” or “pharmacologically acceptable” excipient is a substance that is not biologically harmful or otherwise undesirable. i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “about” denotes a range of +/−10% of a pre-determined value. For example, “about 10” sets a range of 90% to 110% of 10, i.e., 9 to 11.

### DETAILED DESCRIPTION OF THE INVENTION

### I. Introduction

The present invention establishes a highly effective therapeutic strategy involving the delivery of a modified therapeutic RNA using the microbubble/ultrasound scheme. The successful application of this therapeutic strategy has been demonstrated in a Fabry disease (FD) model by supplementing a modified RNA encoding an α-Galactosidase A protein (GLA modRNA). To date, enzyme replacement therapy (ERT) has been among the most effective treatments for patients with Fabry disease (FD). However, new treatment options are still needed to address limitations such as immunogenicity associated with repeated injections leading to reduced therapeutic efficacy of ERT over time. Recent studies in mice have demonstrated the therapeutic potential of nucleoside modified messenger RNA (modRNA) against FD albeit with unresolved toxicity issues associated with the use of lipid nanoparticle as a delivery vector. In this disclosure, the efficacy of a clinically approved method was assessed to mediate in vivo delivery of GLA modRNA encapsulated in microbubbles with or without ectopic exposure to ultrasound waves (sonoporation). It was demonstrated that GLA modRNA can restore the expression and enzymatic activity levels of α-Galactosidase A (αGAL) after transfection in human cardiomyocytes derived from induced pluripotent stem cells (iPSC-CMs) of patients with FD. Importantly. αGAL deficiency in GLA-deficient mice was rescued for at least 42 days as evidenced by significantly upregulated αGAL activity to wide-type levels in the plasma and heart after a single dose of intravenous injection of GLA modRNA encapsulated in microbubbles. The accumulation of glycosphingolipid Globotriaosylceramide (GB3), an αGAL substrate, was also cleared in GLA-deficient mice after GLA modRNA treatment. Furthermore, no detectable increased immunogenicity after modRNA treatment was observed compared to that of ERT. Taken together, these results validate the introduction of a clinically approved, non-toxic and highly efficient protocol for targeted delivery of modRNA with a particular enrichment in the heart after sonoporation, and the therapeutic potential of microbubble encapsulated GLA modRNA to reduce GB3 accumulation in vivo.

### II. General Recombinant Technology

Basic texts disclosing general methods and techniques in the field of recombinant genetics include Sambrook and Russell, Molecular Cloning: A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The sequence of a gene of interest, a polynucleotide encoding a polypeptide of interest, and synthetic polynucleotides or oligonucleotides can be verified after cloning, subcloning, or chemical synthesis using any one of the methods well-known in the relevant field, e.g., the chain termination method for sequencing double-stranded templates of Wallace et al., Gene 16: 21-26 (1981), or an automated sequencing methodology.

### III. Synthesis and Modification of RNA

The RNA molecule used in the method of this invention is designed to encode a functional protein, which is to be recombinantly expressed by a target recipient cell upon delivery of the mRNA into the target cell. An exemplary mRNA for use in this invention encodes a functional human αGAL protein and comprises the polynucleotide sequence set forth in SEQ ID NO:1.

Polynucleotide sequences including RNA or any derivatives or modified versions thereof may be chemically synthesized according to methods known in the pertinent technical field. An RNA molecule can be modified by substitution with one or more nucleotide analogs and/or at the base moiety, sugar moiety, or phosphate backbone, for example, to improve stability of the molecule, hybridization or binding capability, or bioavailability, etc. The polynucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; WO 88/09810) or the blood-brain barrier (see, e.g., WO 89/10134), hybridization-triggered cleavage agents (See, e.g., Krol et al., 1988, BioTechniques 6:958-976) or intercalating agents (see, e.g., Zon, 1988, Pharm. Res. 5:539-549). To this end, the RNA molecule can be conjugated to another molecule for purposes such as tissue/cell targeting, stability, bioavailability, and the like.

The RNA molecules of this invention may be synthesized by standard methods known in the art, e.g., by use of an automated polynucleotide synthesizer (such as are commercially available from Biosearch, Applied Biosystems, etc.). As examples, phosphorothioate polynucleotides may be synthesized by the method of Stein et al. (1988, Nucl. Acids Res. 16:3209), methylphosphonate polynucleotides can be prepared by use of controlled pore glass polymer supports (Sarin et al., 1988, Proc. Natl. Acad. Sci. U.S.A. 85:7448-7451) etc.

### A. In Vitro Synthesis of RNA

In addition to chemical synthetic methods, production of RNA molecules of the present invention can be carried out by recombinant nucleic acid techniques. To obtain a high level of an RNA transcript of a nucleic acid encoding a protein of interest, one typically subclones a polynucleotide encoding the protein into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra.

For instance, the RNA molecules of this invention are in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a polynucleotide sequence encoding a recombinant protein of interest and introducing it into an appropriate vector for in vitro transcription.

In some embodiments, RNA according to the present disclosure comprises a 5′-UTR and/or a 3′-UTR The term “untranslated region” or “UTR” relates to a region in a DNA molecule that is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g., directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g., directly adjacent to the poly(A) sequence.

In some embodiments, the RNA of the present invention comprises a 3′-poly(A) sequence. As used herein, the term “poly(A) sequence” or “poly-A tail” refers to a string of uninterrupted or interrupted adenylate residues located at the 3′-end of an RNA molecule. The RNA molecules of this invention can have a poly(A) sequence attached to the free 3′-end of the RNA by a template-independent RNA polymerase after transcription or a poly(A) sequence encoded by DNA and transcribed by a template-dependent RNA polymerase. It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017). For the present invention, the poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. In some embodiments, a poly(A) sequence is attached during RNA transcription, e.g., during preparation of n vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the RNA of the present invention comprise one or more modified nucleosides as described herein and by methods known in the art. For example, the RNA may include a modified nucleoside in place of at least one (e.g., every) uridine.

When an RNA transcript is produced in an in vitro system in satisfying quantity, it may be purified following the standard nucleic acid purification procedure including size differential filtration and column chromatography. The identity of the RNA molecule may be further verified by methods such as nucleic acid sequence analysis and mass spectrometry.

### B. Modified Nucleotides, Nucleosides and Polynucleotides

When in reference to a nucleotide, nucleoside or polynucleotide (such as the nucleic acids of the invention, e.g., mRNA molecule), the terms “modification” and “modified” describe modification with respect to A, G, U and C ribonucleotides. Generally, these terms are not intended to refer to the ribonucleotide modifications in naturally occurring 5′-terminal mRNA cap moieties.

The modifications may be various distinct modifications. In some embodiments, where the nucleic acid is an mRNA, the coding region, the flanking regions and/or the terminal regions may contain one, two, or more (optionally different) nucleoside or nucleotide modifications. Methods of modifying RNA are known in the field of molecular biology, see, for example, Lui et al., Cell Research 23:1172-86 (2013); Zangi et al., Nature Biotechnology 31:898-907 (2013).

The polynucleotides can include any useful modification, such as to the sugar, the nucleobase, or the inter-nucleoside linkage (e.g., to a linking phosphate/to a phosphodiester linkage/to the phosphodiester backbone). For example, the major groove of a polynucleotide, or the major groove face of a nucleobase may comprise one or more modifications. One or more atoms of a pyrimidine nucleobase (e.g., on the major groove face) may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), e.g., the substitution of the 2′OH of the ribofuranysyl ring to 2′H, threose nucleic acids (TNAs), glycol nucleic acids (GNAs), peptide nucleic acids (PNAs), locked nucleic acids (LNAs) or hybrids thereof).

The polynucleotides of the invention do not substantially induce an innate immune response of a cell into which the polynucleotide (e.g., mRNA) is introduced. Features of an induced innate immune response include 1) increased expression of pro-inflammatory cytokines; 2) activation of intracellular PRRs (RIG-I, MDA5, etc.; and/or 3) termination or reduction in protein translation.

In certain embodiments, it may be desirable for a modified nucleic acid molecule introduced into the cell to be degraded intracellularly. For example, degradation of a modified nucleic acid molecule may be preferable if precise timing of protein production is desired. Thus, in some embodiments, the invention provides a modified nucleic acid molecule containing a degradation domain, which is capable of being acted on in a directed manner within a cell. In other embodiments, a modified polynucleotide introduced to a cell may exhibit reduced degradation in the cell, as compared to an unmodified polynucleotide. In another aspect, the present disclosure provides polynucleotides comprising a nucleoside or nucleotide that can disrupt the binding of a major groove interacting, e.g., binding, partner with the polynucleotide (e.g., where the modified nucleotide has decreased binding affinity to major groove interacting partner, as compared to an unmodified nucleotide).

The modified nucleosides and nucleotides (e.g., building block molecules), which may be incorporated into a polynucleotide (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C1-6 alkyl; optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein): “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges: aminoalkyl, as defined herein; aminoalkoxy, as defined herein: amino as defined herein; and amino acid, as defined herein

Generally. RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid, and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The present disclosure provides for modified nucleosides and nucleotides. As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group. In some embodiments, the nucleosides and nucleotides described herein are generally chemically modified on the major groove face. Exemplary non-limiting modifications include an amino group, a thiol group, an alkyl group, a halo group, or any described herein. The modified nucleotides may by synthesized by any useful method, as described herein (e.g., chemically, enzymatically, or recombinantly to include one or more modified or non-natural nucleosides).

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. These nucleobases can be modified or wholly replaced to provide polynucleotide molecules having enhanced properties, e.g., resistance to nucleases, stability, and these properties may manifest through disruption of the binding of a major groove binding partner. For example, the nucleosides and nucleotides described can be chemically modified on the major groove face. In some embodiments, the major groove chemical modifications can include an amino group, a thiol group, an alkyl group, or a halo group.

In some embodiments, the modified nucleobase is a modified uracil. Exemplary nucleobases and nucleosides having a modified uracil include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1-methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, α-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, and 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl)uridine, and 5-[3-(1-E-propenylamino)uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine (e.g., 5-iodo-cytidine), 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m′A), 2-methyl-adenine, N6-methyl-adenosine, 2-methylthio-N6-methyl-adenosine, N6-isopentenyl-adenosine. 2-methylthio-N6-isopentenyl-adenosine. N6-(cis-hydroxyisopentenyl)adenosine, 2-methylthio-N6-(cis-hydroxyisopentenyl)adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine. N6,N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, α-thio-adenosine, 2′-O-methyl-adenosine. N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, 2′-O-ribosylguanosine, 1-thio-guanosine, 06-methyl-guanosine, 2′-F-ara-guanosine, and 2′-F-guanosine.

In some embodiments, the nucleotide can be modified on the major groove face. For example, such modifications include replacing hydrogen on C-5 of uracil or cytosine with alkyl (e.g., methyl) or halo.

The nucleobase of the nucleotide can be independently selected from a purine, a pyrimidine, a purine or pyrimidine analog. For example, the nucleobase can each be independently selected from adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine. 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

### IV. Pharmaceutical Compositions and Administration

A variety of conditions can be treated by therapeutic approaches that involve introducing a nucleic acid (e.g., a modified RNA of this invention) encoding a functional protein of interest into a target cell such that the coding sequence is transcribed and the protein is produced in the cell for the purpose of correcting a biological deficiency caused or exacerbated by the lack or diminished expression of the protein. For discussions on the application of gene therapy towards the treatment of genetic as well as acquired diseases, see, Miller Nature 357:455-460 (1992); and Mulligan Science 260:926-932 (1993).

### A. Pharmaceutical Compositions

When used for pharmaceutical purposes, the modified RNA encoding a protein of interest is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the modified RNA of the invention. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

### B. Administration

The formulations containing a modified RNA encoding a protein of interest can be delivered to target tissue or organ using any delivery method known to the ordinarily skilled artisan. In some embodiments of the invention, the modified RNA is formulated for subcutaneous, intramuscular, intravenous, or intraperitoneal injection, or for oral ingestion or for nasal inhalation or for topical application. In certain embodiments, a modified RNA of this invention is formulated for administration by injection followed by sonoporation, for example, ultrasound microbubble sonoporation.

The compositions containing the modified RNA of the invention are directly delivered to a target cell. The cell can be provided as part of a tissue, such as heart or lung tissues. The cell can be provided in vivo, ex vivo, or in vitro.

When administered to a patient, effective dosage of the compositions will vary depending on many different factors, including means of administration, target site, physiological state of the patient, and other medicines administered. Thus, treatment dosages will need to be titrated to optimize safety and efficacy. In determining the effective amount of the modified RNA to be administered, the physician should evaluate the particular nucleic acid used, the disease state being diagnosed; the age, weight, and overall condition of the patient, circulating plasma levels, modified RNA toxicities, progression of the disease, and the production of anti-RNA antibodies. The size of the dose also will be determined by the existence, nature, and extent of any adverse side-effects that accompany the administration of a particular RNA. To practice the present invention, doses of a modified RNA ranging from about 0.1 μg-100 mg per patient are typical. Doses generally range between about 0.1 and about 2000 μg per kilogram of body weight, preferably between about 1 and about 1000 μg/kg of bodyweight or about 500 μg/kg bodyweight. In general, the dose equivalent of a modified RNA is from about 7 mg-70 mg for a typical 70 kg patient per injection.

### C. Microbubble-Mediated Gene Transfer

One useful technique of gene delivery in practicing the present invention uses ultrasound-mediated release of genes encapsulated in microbubbles (so-called ultrasound microbubbles). This technique allows for site-specific delivery of a modified RNA with high transfection efficiency and low propensity to cause damage to cells and tissues. Ultrasound is a mechanical vibration with a frequency typically in excess of 20 kHz. It has the advantage of being non-invasive and can be focused into a beam with strong tissue penetration plus being specifically directed to the targeted tissue or organ.

Microbubbles are small bubbles (average diameter <50, 20, or 10 μm) composed of a non-toxic, water-insoluble gas encapsulated with a thin layer of biocompatible material. They have been widely used as ultrasound contrast agents (see, e.g., Inaba Y., Lindner J. R. Molecular imaging of disease with targeted contrast ultrasound imaging. Transl Res. 2012:159:140-148.; Kiessling F., et al. Ultrasound microbubbles for molecular diagnosis, therapy, and theranostics. J Nucl Med. 2012:53:345-348). In recent years, however, they have been evaluated as a new type of drug delivery system. This is based on the fact that ultrasound irradiation of tissue in the presence of microbubbles leads to microbubble oscillation (known as stable cavitation), which generates fluid flow (microflow) in the vicinity of cell membranes sufficient to cause sonoporation and entry of extracellular molecules and particles into the cell. Overall, ultrasound targeted microbubbles have been used to deliver DNA, siRNA (Carson A. R., et al. Ultrasound-targeted microbubble destruction to deliver siRNA cancer therapy. Cancer Res. 2012:72:6191-6199) and drugs (Aryal M., et al. Multiple treatments with liposomal doxorubicin and ultrasound-induced disruption of blood-tumor and blood-brain barriers improve outcomes in a rat glioma model. J Control Release. 2013:169:103-111) to liver, brain, kidney, and other organs for treatment of cancer and cardiovascular disease.

The gas core of a microbubble may comprise one or more gases. For example, the gas core comprises one or more perfluorocarbons. The one or more perfluorocarbons may comprise octafluoropropane (OFP)/perfluoropropane (PFP), decafluorobutane (DFB)/perfluorobutane (PFB), Dodecafluoropentane (DDFP)/perfluoropentane/perflenapent, tetradecafluorohexane/perfluorohexane, or hexadecafluoroheptane/perfluoroheptane, octadecafluorodecalin/perfluorodecalin, or perfluoro(2-methyl-3-pentanone) (PFMP). In some embodiments, the gas core may comprise one of more of the following fluorocarbons: 1,2-(F-alkyl)ethenes; 1,2-bis(F-butyl)ethenes; 1-F-isopropyl, 2-F-hexylethenes 1,2-bis(F-hexyl)ethenes; perfluoromethyldecalins; perfluorodimethyldecalins; perfluoromethyl- and dimethyl-adamantanes, perfluoromethyl-, dimethyl- and trimethyl-bicyclo (3,3,1) nonanes and their homologs. In other examples, the gas core may comprise air, sulfur hexafluoride, or nitrogen. Higher molecular weight gases generally tend to form more stable microbubbles than lower molecular weight gases.

In some embodiments, the microbubble shell may comprise lipids, such as phospholipids, including those for forming microbubbles, nanodroplets, micelles, liposomes, etc., for example, phospholipids comprising diacylglyceride structures, such as phosphatidic acid (phosphatidate) (PA), phosphatidylethanolamine (cephalin) (PE), phosphatidylcholine (lecithin) (PC), phosphatidylserine (PS), and phosphoinositides (e.g., posphatidylinositol (PI), phosphatidylinositol phosphate (PIP), phosphatidylinositol bisphosphate (PIP2) and phosphatidylinositol trisphosphate (PIP3). Further modification of the microbubbles, e.g., PEGylation, may improve anti-flocculation/colloidal stability, anti-immunogenicity, hydrophilicity, biocompatibility, and/or in vivo circulation time/bioavailability of the microbubbles.

Microbubbles of the microbubble compositions describe herein may generally be formed according to any process known in the art. For example, microbubbles may be produced by sonication, by shaking, from high pressure emulsification, or by activating the phase-transition of liquid-core nanodroplets, see, e.g., U.S. Pat. No. 6,113,919: US 2002/0150539; US 2013/0336891; and US 2018/0272012. A composition comprising microbubbles and a modified RNA may be produced by forming microbubbles in the presence of the modified RNA or by simply mixing the modified RNA with pre-formed microbubbles.

Once a composition containing a modified RNA encoding a protein of interest and microbubbles are delivered to target cells, for example, located at a particular anatomic site within a patient's body following a systemic injection (e.g., intravenous injection), ultrasound may be applied to the anatomic site to release the modified RNA and ultimately to express the protein of interest within the target cells at that site, The intensity of the ultrasound waves and the composition of the microbubble shell may influence the effectiveness of the sonoporation. In some embodiments, ultrasound triggering of sonoporation with microbubbles may be performed at frequencies between about 0.1 MHz and about 10 MHz, between about 0.5 MHz and about 5 MHz, or between about 1 MHz and about 3 MHz. In some embodiments, ultrasound triggering may be performed at intensities between about 100 mW/cm² and about 1 kW/cm², between about 100 mW/cm² and about 1 W/cm², between about 300 mW/cm² and about 1 W/cm², between about 500 mW/cm² and about 1 W % cm², between about 700 mW/cm² and about 1 W % cm², between about 1 W/cm2 and about 500 W/cm², between about 1 W/cm² and about 100 W/cm², between about 1 W/cm² and about 100 W/cm², between about 1 W/cm² and about 50 W/cm², between about 1 W/cm² and about 20 W/cm², between about 1 W/cm² and about 10 W/cm², between about 1 W/cm² and about 5 W/cm², or between about 1 W/cm² and about 2 W/cm². In some embodiments, the ultrasound triggering may be performed at a maximal intensity permitted by a regulatory agency (e.g., the FDA), for example, about 720 mW/cm². In some embodiments, the ultrasound triggering may be performed at a duty cycle between about 10% and 100%, between about 20% and about 90%, between about 30% and about 80%, between about 40% and about 70%, or between about 50% and about 60%. In some embodiments, the duty cycle is about 50%. In some embodiments, the mechanical index (MI) of the ultrasound may be between about 0.05 and about 5, between about 0.1 and about 5, between about 0.5 and about 5, between about 1 and about 5, between about 2 and about 5, between about 3 and about between about 4 and about 5. In some embodiments, the ultrasound triggering may be performed at a maximal mechanical index permitted by a regulatory agency (e.g., the FDA), for example, about 1.9. In some embodiments, the ultrasound triggering may be delivered for a time duration of about 10 s-3 min, 10 s-2 min, 10 s-1 min, 10 s-50 s, 10 s-40 s. 10 s-30 s, 30 s-3 min, 30 s-2 min, 30 s-1 min, 30 s-50 s, 30 s-40 s, 1 min-3 min, or 1 min-2 min, or 2 min-3 min.

### V. Additional Therapeutic Agents

Additional known therapeutic agent or agents may be used in combination with a modified mRNA as described herein during the practice of the present invention for the purpose of treating a disease caused or exacerbated by a gene expression deficiency. In such applications, the identity of additional therapeutic agent(s) will depend on the specific disease being treated. For example, if a malignancy is being treated, one or more of these previously known effective anti-cancer therapeutic agents can be administered to patients concurrently with an effective amount of the RNA formulation either together in a single composition or separately in two or more different compositions. They may be used in combination with the active agent of the present invention (the modified RNA in a microbubble composition) to suppress cancer growth, inhibit cancer metastasis, and facilitate remission from the disease.

In this context, various chemotherapeutic agents are known to be effective for use to treat various cancers. As used herein, a “chemotherapeutic agent” encompasses any chemical compound exhibiting suppressive effect against cancer cells, thus useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods disclosed herein also include cytostatic and/or cytotoxic agents.

Exemplary anti-cancer therapeutic agents include alkylating agents such as altretamine, bendamustine, busulfan, carboquone, carmustine, chlorambucil, chlormethine, chlorozotocin, cyclophosphamide, dacarbazine, fotemustine, ifosfamide, lomustine, melphalan, melphalan flufenamide, mitobronitol, nimustine, nitrosoureas, pipobroman, ranimustine, semustine, streptozotocin, temozolomide, thiotepa, treosulfan, triaziquone, triethylenemelamine, trofosfamide, and uramustine; anthracyclines such as aclarubicin, daunorubicin, doxorubicin, epirubicin, idarubicin, mitoxantrone, pirarubicin, valrubicin, and zorubicin: cytoskeletal disruptors (taxanes) such as abraxane, cabazitaxel, docetaxel, larotaxel, paclitaxel, taxotere, and tesetaxel; epothilones such as ixabepilone; histone deacetylase inhibitors such as vorinostat, romidepsin, and inhibitors of topoisomerase I such as belotecan, camptothecin, exatecan, gimatecan, irinotecan, and topotecan; inhibitors of topoisomerase II such as etoposide, teniposide, and tafluposide; kinase inhibitors such as bortezomib, erlotinib, gefitinib, imatinib, vemurafenib, and vismodegib; nucleotide analogs and precursor analogs such as azacitidine, azathioprine, capecitabine, cytarabine, doxifluridine, fluorouracil, gemcitabine, hydroxyurea, mercaptopurine, methotrexate, and tioguanine (formerly thioguanine): peptide antibiotics such as actinomycin and bleomycin; platinum-based agents such as carboplatin, cisplatin, dicycloplatin, oxaliplatin, nedaplatin, and satraplatin; retinoids such as alitretinoin, bexarotene, and tretinoin; and vinca alkaloids and derivatives such as vinblastine, vincristine, vindesine, and vinorelbine.

In one particular embodiment of the present invention, a modified RNA encoding a human αGAL protein in a microbubble composition is administered to a patient suffering from Fabry disease. Galafold® (migalastat) or enzyme replacement therapy (ERT) may be co-administered to the patient.

### VI. Kits

The invention also provides kits for delivering to a target cell a modified RNA encoding a protein of interest and therefore for treating a disease caused or exacerbated by a deficiency in the expression of the native gene encoding the protein in the patient. The kits typically include a container that contains a pharmaceutical composition having an effective amount of a composition comprising microbubbles and a modified RNA encoding the protein of interest. In some cases, a second container is included in the kits to provide a second pharmaceutical composition comprising an effective amount of a second therapeutically active agent known to be effective for treating the same disease. Typically, the kits further includes informational material providing instructions on how to dispense the pharmaceutical composition, including description of the type of patients who may be treated, the schedule (e.g., dose and frequency) and route of administration, and the like.

### Examples

The following examples are provided by way of illustration only and not by way of limitation. Those of skill in the art will readily recognize a variety of non-critical parameters that could be changed or modified to yield essentially similar results.

### Introduction

Fabry disease (FD) is a rare X-linked lysosomal storage disorder due to mutation in the GLA gene that encodes for the lysosomal enzyme α-galactosidase A (αGAL). The deficiency of the enzyme leads to accumulation of its upstream glycosphingolipids, predominately globotriaosylceramide (Gb3), inside lysosomes of all types of cells in the body.^{1,2} The disease mainly affects hemizygous males, and heterozygous females have a spectrum of presentation from asymptomatic to severe disease comparable to that of male counterpart.^{3,4}

There are two types of FD: classic type and late onset type. Classic FD in hemizygous males present early in childhood with symptoms including neuropathic pain, angiokeratoma, gastrointestinal problems, which then progress into cerebrovascular events, renal failure, and hypertrophic cardiomyopathy (HCM) later in life.^{5,6} Late-onset FD patients have residual enzyme activity and present in late adulthood with unexplained renal failure or HCM with minimal involvement of other organs.⁷ The prevalence of FD is around 1 in 40,000 to 117,000, with the majority being late-onset FD (around 7:1 to classic FD), subject to variation in populations.^{3,8,9} In East Asian population, 1 in 875 newborns were screened to have the mutation IVS4+ 919G→A for the cardiac late-onset type.¹⁰

Currently, there is no cure for FD, and the standard of treatment is enzyme replacement therapy (ERT).¹¹ However, ERT is very costly, time consuming and is associated with potential adverse effect. Indeed, cases of severe adverse reactions and immunogenicity have been reported, and antibody development against the protein also renders the treatment less effective.¹¹⁻¹³ Furthermore, reduction of Gb3 level is seen in FD patients after receiving ERT, but does not translate into significant improvement in cardiac function especially with the presence of myocardial fibrosis.¹⁴⁻¹⁷ Indeed, cardiovascular mortality remains to be the major causes of mortality in patients receiving ERT.¹⁸ It is possible that novel approaches with targeted delivery to the heart can further enhance the therapeutic efficacy of treatment of FD related cardiomyopathy.

Recent developments in technology have increased the stability and delivery of mRNA therapy.^{19,20} With modification of RNA nucleotides, there is lower immunogenicity with little or no risk of mutagenesis and has shown to be effective up to 6 weeks after injection in the treatment of FD.²¹⁻²³ However, the clinical application of such nucleoside-modified mRNA (modRNA) is burdened by several concerns, such as the toxicity associated with the use of lipophilic nanoparticles (LNP) as carriers²⁴ and the difficulties in delivering the modRNA to non-liver target organs, such as the heart, after intravenous injection²³

Here, we have demonstrated that GLA modRNA can restore the expression and activity levels of αGAL in human cardiomyocytes derived from patients with FD. We further developed a clinically approved method to mediate in vivo delivery of GL modRNA encapsulated in microbubbles assisting the recapitulation of αGAL activity in the heart, liver and kidney of the GLA-deficient mice particularly after ectopic exposure to ultrasound waves (sonoporation). Besides, the αGAL activity has been maintained at the same levels, if not more, for 42 days after GLA modRNA delivery compared to that after ERT in GLA-deficient mice. The accumulation of Lyso-GB3 is also cleared in GLA-deficient mice after GLA modRNA treatment. Furthermore, no detectable increase in immunogenicity after modRNA treatment is observed compared to that of ERT. Taken together, we have provided a new protocol for targeted delivery of modRNA with a particular enrichment in the heart in vivo. Our findings provide insights into the application of a clinically approved, non-toxic, and highly efficient delivery platform targeting cardiac delivery of modRNA to enhance its therapeutic efficacy in vivo.

### Materials and Methods

Patient-Specific Induced Pluripotent Stem Cell (iPSC) Line of FD

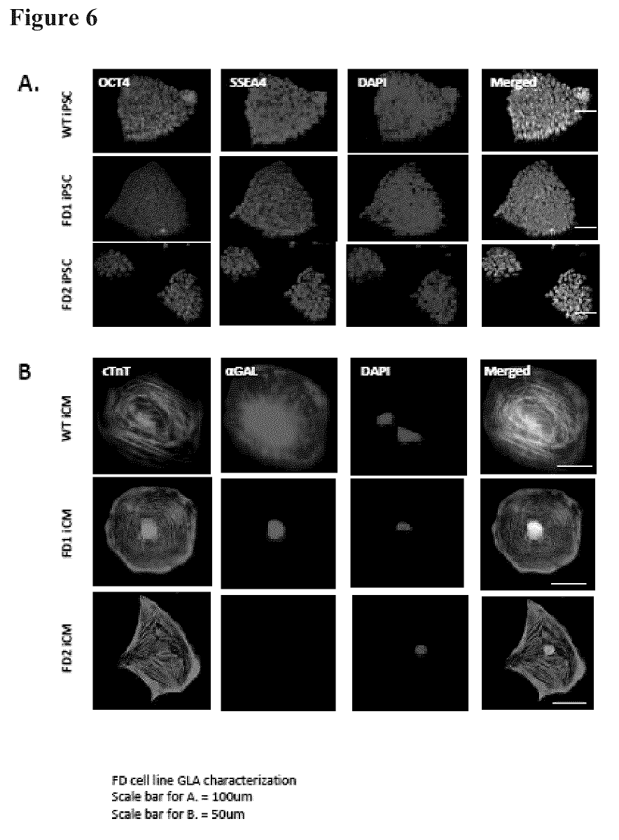

The patient specific iPSC-line was generated from a 65-year old male patient with late-onset cardiac variant of FD carrying IVS4+ 919G→A mutation in the GLA gene as previously described.²⁵ A wide-type male iPSC-line was used as control.²⁶ In brief, the iPSCs were maintained in a serum and feeder free system (MTeSR, Stemcell technologies) and differentiated into cardiomyocytes using the PSC cardiomyocyte differentiation kits (ThermoFisher) (FIG. 6). GLA or GFP-modRNA transfection were introduced into the iPSCs-derived cardiomyocytes (iPSC-CMs) using the Lipofectamine RNAiMAX transfection reagent (ThermoFisher).

### GLA-modRNA Synthesis and Preparation

GLA-modRNA was synthesized according to the methods described previously.²⁷ In brief, plasmids containing open reading frames (ORF) corresponding to human GLA transcript variants 1 was obtained from OriGene (MD, USA). Inserts containing the ORFs of interest was excised and used to template PolyA tail PCRs. RNA was synthesized with the MEGAscriptT7 kit (Ambion) using a ribonucleoside mixture containing 3′-O-Me-m7G(5′)ppp(5′)G cap analog (NEB), adenosine triphosphate and guanosine triphosphate (USB), 5-methylcytidine triphosphate and pseudouridine triphosphate (TriLink). RNA synthesized was purified with Ambion MEGAclear spin columns and then treated with Antarctic Phosphatase (NEB) for 30 min at 37° C. to remove residual 5′-phosphates. The resultant modRNA were re-purified and re-suspended in 10 mM Tris-HCl and 1 mM EDTA; and then the GLA-modRNA were mixed with saline or microbubbles before administration.

SonoVue™ is an echo contrast agent which compose of phospholipids and sulphur hexafluoride to form microbubbles. Each ml of SonoVue™ contains 100-500 million of microbubbles, and their mean diameter is 2.5 μm with >90% of them smaller than 8 μm²⁸. The microbubbles were prepared according to the manufacturer instructions, and 100 μl of the microbubbles was mixed vigorously with 15 μg of the GLA-modRNA (with 20 μl).

### Animal Model of FD

Female C57BL/6 GLA-Knockout (KO) mice (#003535) were purchased from Jackson Laboratory. They were mated with male C57BL/6 wild-type mice to obtain male hemizygous FD mice to evaluate the therapeutic effect of GLA-modRNA. Male C57BL/6 wild-type mice were used as control. All animal experiments were approved by Committee on the Use of Live Animals in Teaching and Research (CULATR) in the University of Hong Kong.

Six months old male GLA-KO mice were used for the in-vivo experiments. Intravenous administration of either 120 μl of saline GLA-modRNA (0.5 μg/g). 120 μl of microbubble coated GLA-modRNA (0.5 μg/g) or 0.5 mg/kg αGAL proteins (R&D Systems, 6146-GH) i.e. enzyme replacement therapy (ERT) with via the tail vein injection. For animals received ultrasound mediated sonoporation, they were anesthetized with 2% isoflurane and exposed to ultrasound at 5 minutes after tail vein injection using Vevo2100™ imaging system (FUJIFILM VisualSonics) as previously described for echocardiography.²⁹ The MX400 probe was positioned at the short axis of the heart and continuously emits ultrasound wave at 18 MHz for about 5 minutes. The papillary muscles were used as landmarks to maintain similar planes of ultrasound exposure between mice. Furthermore, ultrasound mediated sonoporation of microbubble coated GLA-modRNA in liver and kidney were also tested by applications of ultrasound wave at 18 MHz over the liver and kidney for about 5 minutes were tested in two separate groups of FD mice.

### Measurement of αGAL Enzyme Activities and Protein Levels

Supernatants from wide-type and FD iPSC iCMs were corrected at Day The 3 and D15 for measurement of αGAL enzyme activities and protein levels. Plasma enzyme activity of the mice was measured by tail vein blood sampling at Days 3, 7, 21, and 42. Blood samples were collected in EDTA anticoagulant tubes, centrifuged at 4° C. and 2,700×g for 10 min to separate plasma, and stored at −80° C. for later use. The mice were sacrificed at the Day 3 or Day 42, and tissue lysates (heart, liver, kidney) were extracted for measurement of enzyme activity and protein level. Enzyme activity was measured with αGAL enzyme activity assay kit (ab239716, Abcam, Cambridge, MA), and protein concentration was measured with Perce™ BCA protein assay kit (Thermo Fisher Scientific).

### Gene Expression Analysis

RNA was extracted from different tissues using TRIzol™ reagent (Thermo Fisher Scientific) and reverse-transcribed into cDNA with the Qiagen™OneStep RT-PCR kit. The gene expression was evaluated by real time PCR analysis using QuantiTect™ SYBR™ Green reagent (Qiagen) and gene specific oligonucleotides with StepOnePlus™ Real-Time PCR system (Applied Biosystems). The relative expression levels of the genes of interest were determined using the delta-delta CT-based method, in which the expressions of GAPDH were used as internal references.

### Immunohistochemistry

Immunostaining of αGAL protein was performed on formalin-fixed paraffin embedded sections after antigen retrieval. The slides were stained with primary anti-GLA antibody (PA5-27349, ThermoFisher) and HRP-conjugated secondary antibodies and visualized with DAB substrate kits (ThermoFisher). After counterstaining with hematoxylin, the slides were mounted with Histomount Mounting Solution (Thermofisher) for light microscopy imaging.

### Blood Biochemistry, Antibody, Cytokines and Immune Cell Flow Cytometry

Plasma level of alanine transaminase (A526-120, Teco Diagnostics), aspartate transaminase (A560-400, Teco Diagnostics), and lactate dehydrogenase (CyQUANT™ LDH Cytotoxicity Assay, C20300, Invitrogen) were measured with pre-designed kits. Plasma levels of anti-hGLA antibody was measured with ELISA as previously mentioned.²³ Plasma quantification of cytokines was performed with LEGENDplex™ Mouse Inflammation Panel (BioLegend, #740446). Isolated splenocytes were measured for their immune cell composition with anti-mouse CD4 and CD8 antibodies (BioLegend, #100405, #100711), anti-mouse CD11 b and Ly-6c antibodies (BioLegend, #101205, #128015), anti-mouse CD49b and Nk1.1 antibodies (BioLegend, #108909, #108705), and 7-AAD viability staining solution (#130-111-568, MiltenylBiotec) before quantifying with CytoFLEX flow cytometer (Beckman Coulter).

### Mass Spectrometry

Plasma lyso-Gb3 levels were quantified using protocols previously described.³⁰ In brief, lyso-Gb3 was extracted from 100 μl of plasma using 1 ml of acetone:methanol (1:1 vol/vol), in which, 2 ng/ml di-methyl psychosine (2 ng/ml) (Avanti Polar Lipids Inc.) was added as internal standard. The mixture was then vortexed for 20 min and sonicated for 15 min at room temperature. After centrifugation at 16,000 g, supernatants were transferred and evaporated on a rotational evaporator at room temperature. Samples were reconstituted in 100 μl of methanol and subjected to LC-MS/MS analysis. ACQUITY UPLC system (Waters Corporation, Milford, MA) was used for the separation of Lyos-Gb3 before quantitative tandem mass spectrometry analysis based on the following parameter: (column: Acquity UPLC BEH C18, Waters Corp.: Length: 50 mm; internal diameter; 2.1 mm, particle diameter: 1.7 μM; column temperature 40° C.; weak wash solvent: 0.1% TFA; strong wash solvent: IPA:ddH2O:MeOH:can; mobile phase A: 0.1% FA; mobile phase B: 100% MeOH; Gradient: Gradient parameters 1; flow rate: 0.6 ml/min; injection volume; 5 μl; injection mode: partial loop; auto-sampler temperature: 10° C.). The UPLC system was coupled to a Xevo TQ-S (Waters Ltd., UK) mass spectrometer using the multiple reaction monitoring mode and the concentration of lyso-Gb3 was evaluated using the TargetLynx 4.1 software (Waters). Dimethylpsychosine was used as internal standard for lyso-Gb3. Calibration curves were linear the origin was excluded.

### Statistical Analysis

All data are expressed as mean f SEM from at least 3 sets of independent experiments on at least 3 sets of biological replicates unless specified elsewhere. Statistical analyses were performed using Prizm software (GraphPad Inc.). Comparison of parameters between different groups was performed using the Mann-Whitney U test or 1-way analysis of variance with Tukey's test, as appropriate. Differences were considered statistically significant at a level of p<0.05.

### Results

Expression of αGAL after Transfection of GLA-modRNA in FD Patient-Specific iPSC-CMs In-Vitro

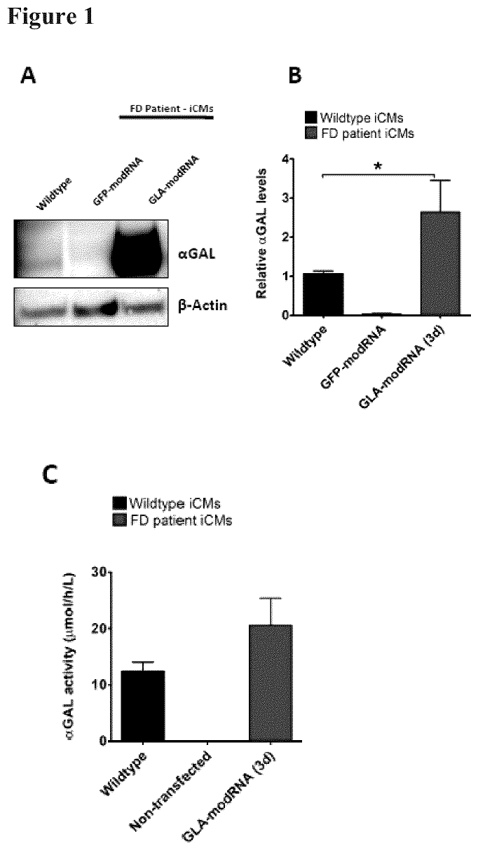

In this study, we generated nucleoside modified GLA-modRNA by replacing cytidine with methyl-cytidine and uridine with pseudouridine as previously reported. The efficacy of the GL4-modRNA was first evaluated with iPSC-CMs with a IVS4+ 919G→A mutation. Immunostaining for OCT4 and SSEA4 confirmed expression of pluripotency markers in iPSCs derived from healthy (wild-type) and FD patients (FIG. 6A). Following the differentiation of iPSCs into cardiomyocytes, immunostaining for αGAL further confirmed markedly reduced expression in cardiomyocytes derived from FD patient-specific iPSCs (FIG. 6B). After a single transfection of GLA-modRNA in iPSC-CMs, Western blot and enzyme activity analysis of the supernatants respectively revealed that αGAL protein expression (FIGS. 1A and 1B) and activity (FIG. 1C) were significantly greater at Day 3 after transfection in FD patient-specific iPSC-CMs compared with wide-type iPSC-CMs, and untransfected FD iPSC-iCMs.

### Ultrasound Mediated Sonoporation Enhanced Targeted Tissue Delivery GLA-modRNA In-Vivo

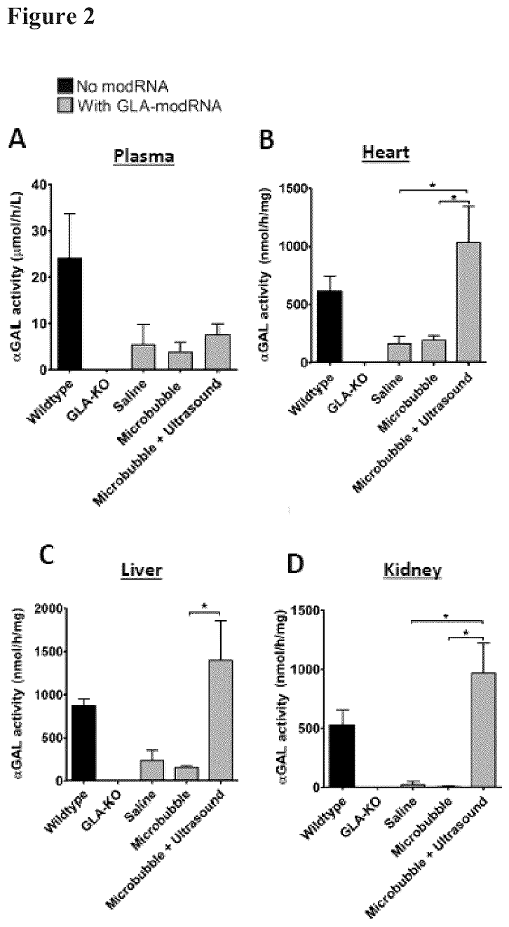

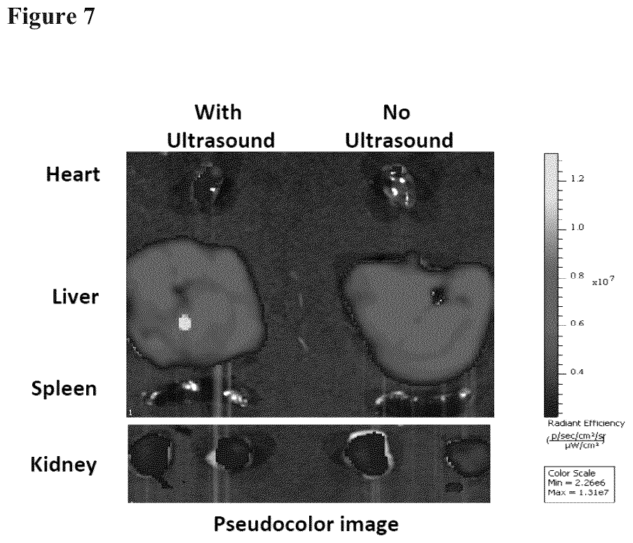

Next, we investigated whether the application of ultrasound at different sites can enhance the local tissue delivery of GLA-modRNA using microbubbles as carriers. For proof of concept. 50 ug GFP modRNA encapsulated by microbubbles was intravenously injected into C57Bl/6 mice and different vascularized organs including the heart, liver, spleen and kidneys were subjected for in vivo imaging after injection. Indeed, the heart was found more effectively labeled at day 1 after cardiac sonoporation (FIG. 7). Moreover, 0.5 ug/g body weight GLA-modRNA was intravenously injected into GLA-deficient mice, and the αGAL enzyme activities of the plasma, heart, liver and kidney were measured at day 3 after modRNA delivery. Wild type and untreated GLA-deficient mice were respectively included as positive and negative controls. The plasma enzymatic activities after the delivery of GLA-modRNA in the form of saline, microbubble with or without cardiac sonoporation did not show significant difference (all P>0.05, FIG. 2A). On the other hand, cardiac sonoporation significantly increased the myocardial αGAL enzyme activity in mice injected with microbubble encapsulated GLA-modRNA compared to the other groups (FIG. 2B, all P<−0.05). Similarly, liver and kidney sonoporation also significantly increased the local αGAL enzymatic activity in mice injected with microbubble encapsulated GLA-modRNA compared to the other groups (FIGS. 2C and 2D, all P<0.05). These findings demonstrated that ultrasound mediated sonoporation can enhance the local targeted delivery of microbubble coated GLA-modRNA with increased local αGAL enzymatic activities in GLA-deficient mice in vivo.

### Targeted Cardiac Delivery of Microbubble Encapsulated GLA-modRNA Restores αGAL Activity and Function in Mouse Model of FD In-Vivo

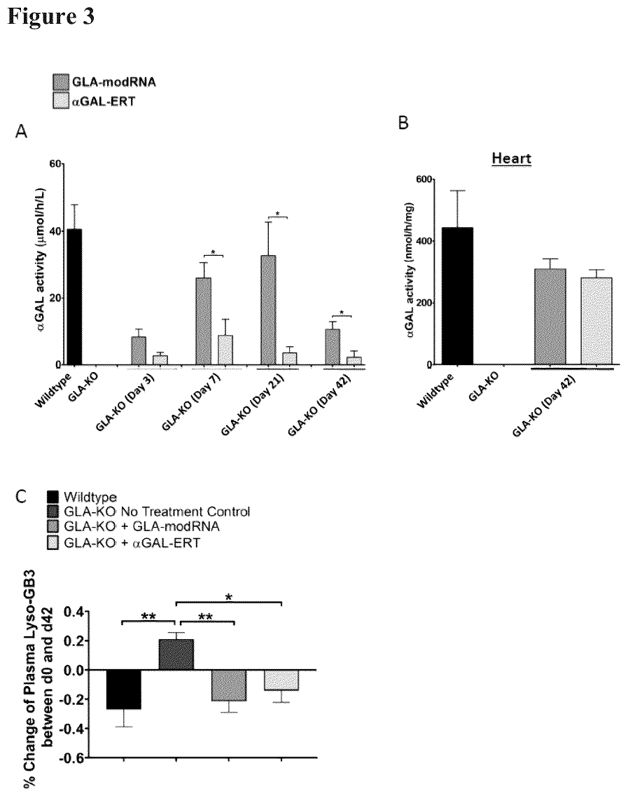

The potential therapeutic efficacy of targeted cardiac delivery of microbubble coated GLA-modRNA was compared with αGAL ERT in a mouse model of FD in-vivo. In both groups, a single intravenous administration of microbubble coated GLA-modRNA or αGAL ERT were administrated. Wild type and untreated GLA-deficient mice were respectively included as positive and negative controls. Serial plasma samples were collected at regular intervals (Days 3, 7, 21, 42 after injection), and mice were sacrificed at Day 42 after injection. As shown in FIG. 3A, the plasma αGAL enzymatic activity levels were significantly higher at Days 7, 21, and 42 in the microbubble coated GLA-modRNA with cardiac sonoporation as compared to that of the ERT group (all P<0.05). At Day 42, the myocardial αGAL enzymatic activity in mice after receiving microbubble coated GL4-modRNA with cardiac sonoporation was comparable to that receiving ERT; and was not significantly lower than that of the wide-type mice (FIG. 3B, all P>0.05).

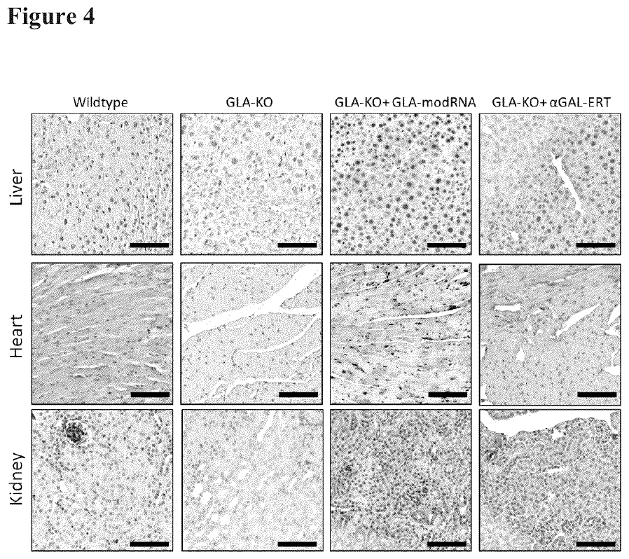

To date, accumulation of glycosphingolipids, such as lyso-Gb3, is a hallmark of FD progression.^{31,32} As the lyso-Gb3 level has been shown to be a reliable biomarker for monitoring FD progression,³¹ we have also evaluated the changes of plasma lyso-Gb3 levels before and after treatment. As shown in FIG. 3C, in the absence of any treatment, the lyso-Gb3 levels were significantly increased in the GLA-KO mice compared to that of the wild-type group (P<0.01). On the other hand, treatment with microbubble coated GLA-modRNA with sonoporation or ERT significantly decreased the plasma lyso-Gb3 levels compared with untreated GLA-KO mice at Day 42 after administration (FIG. 3C, P<0.05), and the lyso-Gb3 levels of the treated mice were comparable to that of the wild-type control group (FIG. 3C, P>0.05). Furthermore, the expression levels of αGAL proteins in different groups were also confirmed by immunohistochemistry. As shown in FIG. 4, αGAL proteins were detected in the liver, heart and kidney of GLA-deficient mice at Day 42 after a single injection of microbubble coated GLA-modRNA with sonoporation or with ERT. Taken together, microbubble encapsulated GLA-modRNA with sonoporation contributed to a significantly higher circulating αGAL enzymatic activity than ERT and was as effective as ERT in decreasing the plasma lyso-Gb3 levels, coupled with increased myocardial αGAL enzymatic activity and protein expression in GLA-deficient mice after a single administration.

Lack of Immunogenicity with Microbubble Coated GLA-modRNA

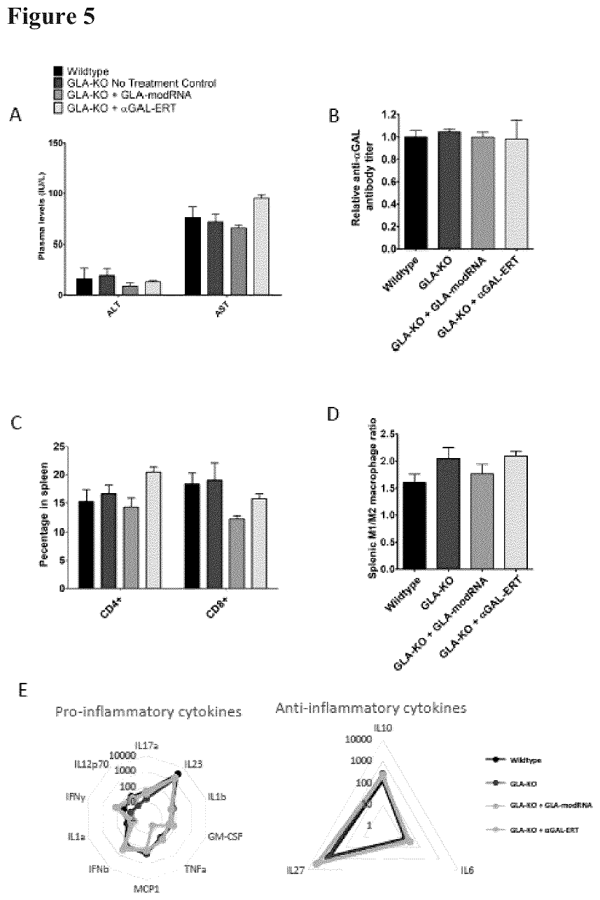

Lastly, the potential toxicity and immunogenicity associated with the use of microbubble coated GLA-modRNA was also evaluated at Day 42 in vivo. As demonstrated in FIG. 5A, there were no significant differences m the plasma levels of alanine transaminase, aspartate transaminase and lactate dehydrogenase after GL-modRNA treatment in GLA-deficient mice compared to that of the wild type control or ERT group (P>0.05), indicating normal liver function. Besides, there was also no significant difference in the plasma levels of anti-αGAL neutralizing antibody after GLA-modRNA administration compared to that of the wildtype control or ERT group (FIG. 5B, P>0.05). Moreover, flow cytometric analysis showed similar percentages of CD4⁺ and CD8⁺ T cells (FIG. 5C, P>0.05) as well as a comparable M1 to M2 macrophage ratio (FIG. 5D, P>0.05) in the spleen after GLA-modRNA treatment. The plasma inflammatory cytokine levels were also not significantly different after GLA-modRNA treatment (FIG. 5E, P>0.05). Altogether, these results demonstrate that systemic administration of microbubble coated GL4-modRNA did not induce any noticeable toxicity, immunogenicity or inflammatory response in vivo.

### DISCUSSION

In this study, we demonstrated the possibility of using a clinically approved tool to enhance local delivery of GLA-modRNA encapsulated in microbubbles after ectopic exposure to sonoporation in a FD mouse model. Compared to the conventional ERT approach through administration of human αGAL protein, administration of microbubble encapsulated GLA-modRNA with and without ultrasound significantly increased the αGAL enzymatic activities in plasma without obvious immune reactions. In addition, microbubbles encapsulated GLA-modRNA also exerted a longer duration of action compared to the ERT approach. Specifically, the circulating enzymatic activity of αGAL remained detectable until 42 days after a single injection of modRNA, indicating a longer half-life of the product. In contrast, ERT usually requires injection every two weeks to maintain a critical circulating level of αGAL protein^{41,41}. Although the intracellular enzymatic activities in the heart were similar between the modRNA and ERT approaches cells may not maintain a high protein level of αGAL in homeostasis. Indeed, it has been reported that only a small amount of αGAL protein (5-10% concentration) is sufficient to clear the substrates and prevent pathogenic accumulation¹. Thus, we speculate that the intracellular enzymatic activity level may not differ among different administration methods as cells only maintain a residual level of enzymes capable of clearing Gb3 while secreting the excessive proteins into the circulation.

The principle of ultrasound-assisted delivery method relies on the sonoporation, i.e. the bursting of microbubbles when exposed to ultrasound waves. When microbubbles travel through the organs under ultrasound exposure, these bubbles burst and release the modRNA to the proximity. With higher concentration of modRNA in the vicinity, the organ could uptake more modRNA, leading to more efficient organ-specific targeted delivery. In addition, sonoporation may induce transient endothelial damages that could facilitate the uptake of mRNA^{33,34}. Microbubbles may also protect the modRNA from or delay the degradation inside the blood vessels. Although modRNA would mostly be delivered to the liver through intravenous injection, the microbubble encapsulated modRNAs are likely trapped inside the microvasculature and slowly releases its modRNA content to the surrounding hepatocytes. After uptake, these hepatocytes can translate the modRNA into proteins and excrete them into the circulation for uptake from other cell types such as cardiomyocytes and renal tubular cells^{23,25}.

Comparing to conventional ERT, administration of microbubble encapsulated modRNA also increased the hepatic production of αGAL protein, which may allow for a longer interval between injections before the αGAL protein levels became suboptimal. Previous studies have shown that the efficacy of modRNA is around 4 weeks without the use of microbubbles as carriers^{23,36}. With microbubble encapsulation, our data showed that a relatively higher level of αGAL protein was still detected in the plasma at 42 days after a single injection. Furthermore, one of the major concerns for current ERT is the inconvenience of treatment and extremely high cost for patients, as the administrations need to be repeated every 2-3 weeks for life maintenance.^{19,20} The use of microbubbles with and without ultrasound provides alternatives for patients with potentially extended injection intervals. As ultrasound-dependent sonoporation has shown to significantly increase the enzymatic activity in local tissues, it could be used for more acute settings as a tissue-specific therapy; while delivery of microbubble encapsulated modRNA without sonoporation could be used for maintaining a long-term enzyme baseline in the circulation.

Immunogenic reaction has been another concern for many patients, as the neutralizing antibodies generated were shown to decrease treatment effectiveness^{13,37,38}. In our study, there was no significant inflammatory reaction or organ damage demonstrated after the delivery of microbubble encapsulated modRNA in vivo compared to the control group even at 6 weeks after injection. On the other hand, as documented by previous studies, the stress-related gene expression has shown to be increased after αGAL-ERT. Although this could be due to the immune responses against exogenous supply of αGAL protein, the employment of organism's own machinery in the production of αGAL enzyme from modRNA therapy has proved to be a safer option. The immune system usually requires sensitization from non-self proteins before mounting more effective secondary responses or rejections against the foreign antigen, which has been demonstrated in repeated injection of other non-self particles³⁹. Nevertheless, we did not examine the immunogenicity of microbubble encapsulated modRNA following multiple injections that would warrant further investigations Another limitation of the current study is the lack of severe cardiac presentations in the FD mouse model. The lack of human disease phenotypes in GLA-KO mice have been well documented, and the reported phenotypes, if any, were either inconclusive or controversial^{35,40}. Thus, the detection of Gb3 clearance has been utilized to monitor disease progression and improvement in mice for therapeutic discovery, even though one may not be able to directly demonstrate the improvement of symptoms or inhibition of disease progression based on phenotype. Future studies should examine the therapeutic potential of microbubble encapsulated GLA-modRNA in large animal models of Fabry disease for better clinical translation. Taken together, we demonstrate a clinically approved strategy to enhance the efficacy and tissue specificity through intravenous delivery of microbubble encapsulated GLA-modRNA controlled by sonoporation. Our study also provides clinically relevant insights into novel delivery strategy targeting mRNA therapy.

All patents, patent applications, and other publications, including GenBank Accession Numbers or similar sequence identification numbers, cited in this application are incorporated by reference in the entirety of their contents for all purposes.

### REFERENCES

1. Zarate Y A, Hopkin R J. Fabry's disease. Lancet. 2008; 372:1427-1435. doi: 10.1016/50140-6736(08)61589-5
2. Germain D P. Fabry disease. Orphanet J Rare Dis. 2010; 5:30. doi: 10.1186/1750-1172-5-30
3. Desnick R J I, Yiannis A.; Eng, Christine M. α-Galactosidase A Deficiency: Fabry Disease. In: Valle D L A, Stylianos; Ballabio, Andrea; Beaudet, Arthur L.; Mitchell, Grant A., ed. The Online Metabolic and Molecular Bases of Inherited Disease. New York: McGraw-Hill; 2001:3733-3774.
4. Wilcox W R, Oliveira J P, Hopkin R J, Ortiz A, Banikazemi M, Feldt-Rasmussen U, Sims K, Waldek S, Pastores G M, Lee P, et al. Females with Fabry disease frequently have major organ involvement: lessons from the Fabry Registry. Mol Genet Metab. 2008; 93:112-128. doi: 10.1016/j.ymgme.2007.09.013
5. Hopkin R J, Bissler J, Banikazemi M, Clarke L, Eng C M, Germain D P, Lemay R, Tylki-Szymanska A, Wilcox W R. Characterization of Fabry disease in 352 pediatric patients in the Fabry Registry. Pediatr Res. 2008; 64:550-555. doi: 10.1203/PDR.0b013e318183f132
6. MacDermot K D, Holmes A, Miners A H. Anderson-Fabry disease: clinical manifestations and impact of disease in a cohort of 98 hemizygous males. J Med Genet. 2001; 38:750-760. doi: 10.1136/jmg.38.11.750
7. Michaud M, Mauhin W, Belmatoug N, Garnotel R, Bedreddine N, Catros F, Ancellin S, Lidove O, Gaches F. When and How to Diagnose Fabry Disease in Clinical Practice. Am J Med Sci. 2020; 360:641-649. doi: 10.1016/j.amjms.2020.07.011
8. Meikle P J, Hopwood J J, Clague A E, Carey W F. Prevalence of lysosomal storage disorders. JAMA. 1999; 281:249-254. doi: 10.1001/jama.281.3.249
9. Spada M, Pagliardini S, Yasuda M, Tukel T, Thiagarajan G, Sakuraba H, Ponzone A, Desnick R J. High incidence of later-onset fabry disease revealed by newborn screening. Am J Hum Genet. 2006; 79:31-40. doi: 10.1086/504601
10. Chien Y H, Lee N C, Chiang S C, Desnick R J, Hwu W L. Fabry disease: incidence of the common later-onset alpha-galactosidase A IVS4+ 919G->A mutation in Taiwanese newborns—superiority of DNA-based to enzyme-based newborn screening for common mutations. Mol Med. 2012; 18:780-784. doi: 10.2119/molmed.2012.00002
11. Schiffmann R, Kopp J B, Austin H A, 3rd, Sabnis S, Moore D F, Weibel T, Balow J E, Brady R O. Enzyme replacement therapy in Fabry disease: a randomized controlled trial. JAMA. 2001; 285:2743-2749. doi: 10.1001/jama.285.21.2743
12. Eng C M, Guffon N, Wilcox W R, Germain D P, Lee P, Waldek S, Caplan L, Linthorst G E, Desnick R J, International Collaborative Fabry Disease Study G. Safety and efficacy of recombinant human alpha-galactosidase A replacement therapy in Fabry's disease. N Engl J Med. 2001; 345:9-16. doi: 10.1056/NEJM200107053450102
13. Banikazemi M, Bultas J, Waldek S, Wilcox W R, Whitley C B, McDonald M, Finkel R, Packman S, Bichet D G, Warnock D G, et al. Agalsidase-beta therapy for advanced Fabry disease: a randomized trial. Ann Intern Med. 2007; 146:77-86. doi: 10.7326/0003-4819-146-2-200701160-00148
14. Vedder A C, Linthorst G E, Houge G, Groener J E, Ormel E E, Bouma B J, Aerts J M, Hirth A, Hollak C E. Treatment of Fabry disease: outcome of a comparative trial with agalsidase alfa or beta at a dose of 0.2 mg/kg. PLoS One. 2007; 2:e598. doi: 10.1371/journal.pone.0000598
15. Hughes D A, Elliott P M, Shah J, Zuckerman J, Coghlan G, Brookes J, Mehta A B. Effects of enzyme replacement therapy on the cardiomyopathy of Anderson-Fabry disease: a randomised, double-blind, placebo-controlled clinical trial of agalsidase alfa. Heart. 2008; 94:153-158. doi: 10.1136/hrt.2006.104026
16. Schaefer R M, Tylki-Szymanska A, Hilz M J. Enzyme replacement therapy for Fabry disease: a systematic review of available evidence. Drugs. 2009; 69:2179-2205. doi: 10.2165/11318300-000000000-0000
17. Weidemann F, Niemann M, Breunig F, Herrmann 5, Beer M, Stork S, Voelker W, Ertl G, Wanner C, Strotmann J. Long-term effects of enzyme replacement therapy on fabry cardiomyopathy: evidence for a better outcome with early treatment. Circulation. 2009; 119:524-529. doi: 10.1161/CIRCULATIONAHA.108.794529
18. Ferrari G, Reisin R, Kisinovsky I, Neumann P, Dragonetti L, Caceres G, Choua M, Rozenfeld P, Marchesoni C, Finn V, et al. Major cardiovascular adverse events in Fabry disease patients receiving agalsidase alfa. Medicina (B Aires). 2021; 81:173-179.
19. van der Veen S J, Hollak C E M, van Kuilenburg A B P, Langeveld M. Developments in the treatment of Fabry disease. J inherit Metab Dis. 2020; 43:908-921. doi: 10.1002/jimd.12228
20. Azevedo O, Gago M F, Miltenberger-Miltenyi G, Sousa N, Cunha D. Fabry Disease Therapy: State-of-the-Art and Current Challenges. Int J Mol Sci. 2020; 22. doi: 10.3390/ijms22010206
21. Kariko K, Muramatsu H, Welsh F A, Ludwig J, Kato H, Akira S, Weissman D. Incorporation of pseudouridine into mRNA yields superior nonimmunogenic vector with increased translational capacity and biological stability. Mol Ther. 2008; 16:1833-1840. doi: 10.1038/mt.2008.200
22. DeRosa F S, L.; Shen, Y.; Huang, Y.; Pan, J.; Xie, H.; Yahalom, B.; Heartlein, M. W. Improved Efficacy in a Fabry Disease Model Using a Systemic mRNA Liver Depot System as Compared to Enzyme Replacement Therapy. Molecular therapy: the journal of the American Society of Gene Therapy. 2019; 27:878-889.
23. Zhu X. Yin L, Theisen M, Zhuo J, Siddiqui S, Levy B, Presnyak V, Frassetto A, Milton J, Salerno T, et al. Systemic mRNA Therapy for the Treatment of Fabry Disease: Preclinical Studies in Wild-Type Mice, Fabry Mouse Model, and Wild-Type Non-human Primates. Am J Hum Genet. 2019; 104:625-637. doi: 10.1016/j.ajhg.2019.02.003
24. Dhiman N, Awasthi R, Sharma B, Kharkwal H, Kulkarni G T. Lipid Nanoparticles as Carriers for Bioactive Delivery. Front Chem. 2021; 9:580118. doi: 10.3389/fchem.2021.580118
25. Kotevski L, Gligorovski L, Dimitrovski M. Example of assisted mechanical ventilation in obstructive syndrome of UIRC. Godisen zbomik no Medicinskiotfakultet vo Skopje. 1979; 25:181-189.
26. Siu C W, Lee Y K, Ho J C, Lai W H, Chan Y C, Ng K M, Wong L Y, Au K W, Lau Y M, Zhang J, et al. Modeling of lamin A/C mutation premature cardiac aging using patient-specific induced pluripotent stem cells. Aging. 2012; 4:803-822. doi: 10.18632/aging.100503
27. Zangi L, Lui K O, von Gise A, Ma Q, Ebina W, Ptaszek L M, Spater D, Xu H, Tabebordbar M, Gorbatov R, et al. Modified mRNA directs the fate of heart progenitor cells and induces vascular regeneration after myocardial infarction. Nature biotechnology. 2013; 31:898-907. doi: 10.1038/nbt.2682
28. Schneider M. Characteristics of SonoVuetrade mark. Echocardiography. 1999; 16:743-746. doi: 10.1111/j.1540-8175.1999.tb00144.x
29. Zacchigna S, Paldino A, Falcao-Pires I, Daskalopoulos E P, Dal Ferro M, Vodret S, Lesizza P, Cannata A, Miranda-Silva D, Lourenco A P, et al. Towards standardization of echocardiography for the evaluation of left ventricular function in adult rodents: a position paper of the ESC Working Group on Myocardial Function. Cardiovasc Res. 2021; 117:43-59. doi: 10.1093/cvr/cvaa110
30. Heywood W E, Doykova I, Spiewaka J, Hallqvista J, Mills K, Nowak A. Global glycosphingolipid analysis in urine and plasma of female Fabry disease patients. Biochimica et Biophysica Acta (BBA)—Molecular Basis of Disease. 2019; 1865:2726-2735. doi: 10.1016/j.bbadis.2019.07.005
31. Sakuraba H, Togawa T, Tsukimura T, Kato H. Plasma lyso-Gb3: a biomarker for monitoring fabry patients during enzyme replacement therapy. Clin Exp Nephrol. 2018; 22:843-849. doi: 10.1007/s10157-017-1525-3
32. Maruyama H, Miyata K, Mikame M, Taguchi A, Guili C, Shimura M, Murayama K, Inoue T, Yamamoto S, Sugimura K, et al. Effectiveness of plasma lyso-Gb3 as a biomarker for selecting high-risk patients with Fabry disease from multispecialty clinics for genetic analysis. Genet Med. 2019; 21:44-52. doi: 10.1038/gim.2018.31
33. Karshafian R, Bevan P D, Williams R, Samac S, Burns P N. Sonoporation by ultrasound-activated microbubble contrast agents: effect of acoustic exposure parameters on cell membrane permeability and cell viability. Ultrasound Med Biol. 2009; 35:847-860. doi: 10.1016/j.ultrasmedbio.2008.10.013
34. Wang M, Zhang Y, Cai C L, Tu J, Guo X S, Zhang X. Sonoporation-induced cell membrane permeabilization and cytoskeleton disassembly at varied acoustic and microbubble-cell parameters. Sci Rep. 2018; 8. doi: 10.1038/s41598-018-22056-8
35. Li C, Ziegler R J, Cherry M, Lukason M, Desnick R J, Yew N S, Cheng S H. Adenovirus-transduced lung as a portal for delivering alpha-galactosidase A into systemic circulation for Fabry disease. Mol Ther. 2002; 5:745-754. doi: 10.1006/mthe.2002.0605
36. Magadum A, Kaur K, Zangi L. mRNA-Based Protein Replacement Therapy for the Heart. Mol Ther. 2019; 27:785-793. doi: 10.1016/j.ymthe.2018.11.018
37. Eng C M, Banikazemi M, Gordon R E, Goldman M, Phelps R, Kim L, Gass A, Winston J, Dikman S, Fallon I T, et al. A phase 1/2 clinical trial of enzyme replacement in fabry disease: pharmacokinetic, substrate clearance, and safety studies. Am J Hum Genet. 2001; 68:711-722. doi: 10.1086/318809
38. Schiffmann R, Ries M, Timmons M, Flaherty J T, Brady R O. Long-term therapy with agalsidase alfa for Fabry disease: safety and effects on renal function in a home infusion setting. Nephrol Dial Transplant. 2006; 21:345-354. doi: 10.1093/ndt/gfi152
39. Laverman P, Carstens M G, Boerman O C, Dams E T, Oyen W J, van Rooijen N, Corstens F H, Storm G. Factors affecting the accelerated blood clearance of polyethylene glycol-liposomes upon repeated injection. J Pharmacol Exp Ther. 2001; 298:607-612.
40. Elsea S H, Lucas R E. The mousetrap: what we can learn when the mouse model does not mimic the human disease. ILARJ. 2002; 43:66-79. doi: 10.1093/ilar.43.2.66
41. Schiffmann R, Goker-Alpan O, Holida M, Giraldo P. Barisoni L, Colvin R B, Jennette C J, Maegawa G, Boyadjiev S A, Gonzalez D, et al. Pegunigalsidase alfa, a novel PEGylated enzyme replacement therapy for Fabry disease, provides sustained plasma concentrations and favorable pharmacodynamics: A 1-year Phase 1/2 clinical trial. J Inherit Metab Dis. 2019; 42:534-544. doi: 10.1002/jimd.12080
42. Hennermann J B, Arash-Kaps L, Fekete G, Schaaf A, Busch A, Frischmuth T. Pharmacokinetics, pharmacodynamics, and safety of moss-aGalactosidase A in patients with Fabry disease. J Inherit Metab Dis. 2019; 42:527-533. doi: 10.1002/jimd.12052

SEQ ID NO: 1 (coding sequence for α-galatocidase):
Ttggaccctcgtacagaagctaatacgactcactatagggaaataagaga
gaaaagaagagtaagaagaaatataagagccaccatgcagctgaggaacc
cagaactacatctgggctgcgcgcttgcgcttcgcttcctggccctcgtt
tcctgggacatccctggggctagagcactggacaatggattggcaaggac
gcctaccatgggctggctgcactgggagcgcttcatgtgcaaccttgact
gccaggaagagccagattcctgcatcagtgagaagctcttcatggagatg
gcagagctcatggtctcagaaggctggaaggatgcaggttatgagtacct
ctgcattgatgactgttggatggctccccaaagagattcagaaggcagac
ttcaggcagaccctcagcgctttcctcatgggattcgccagctagctaat
tatgttcacagcaaaggactgaagctagggatttatgcagatgttggaaa
taaaacctgcgcaggcttccctgggagttttggatactacgacattgatg
cccagacctttgctgactggggagtagatctgctaaaatttgatggttgt
tactgtgacagtttggaaaatttggcagatggttataagcacatgtcctt
ggccctgaataggactggcagaagcattgtgtactcctgtgagtggcctc
tttatatgtggccctttcaaaagcccaattatacagaaatccgacagtac
tgcaatcactggcgaaattttgctgacattgatgattcctggaaaagtat
aaagagtatcttggactggacatcttttaaccaggagagaattgttgatg
ttgctggaccagggggttggaatgacccagatatgttagtgattggcaac
tttggcctcagctggaatcagcaagtaactcagatggccctctgggctat
catggctgctcctttattcatgtctaatgacctccgacacatcagccctc
aagccaaagctctccttcaggataaggacgtaattgccatcaatcaggac
cccttgggcaagcaagggtaccagcttagacagggagacaactttgaagt
gtgggaacgacctctctcaggcttagcctgggctgtagctatgataaacc
ggcaggagattggtggacctcgctcttataccatcgcagttgcttccctg
ggtaaaggagtggcctgtaatcctgcctgcttcatcacacagctcctccc
tgtgaaaaggaagctagggttctatgaatggacttcaaggttaagaagtc
acataaatcccacaggcactgttttgcttcagctagaaaatacaatgcag
atgtcattaaaagacttactttaggctgccttctgcggggcttgccttct
ggccatgcccttcttctctcccttgcacctgtacctcttggtctttgaat
aaagcctgagtaggaag

1. A method for delivering to a target cell a nucleic acid encoding a protein of interest, comprising:
(i) contacting the cell a composition comprising microbubbles and the nucleic acid encoding the protein of interest; and
(ii) applying ultrasound to the target cell, thereby introducing the nucleic acid into the target cell,
wherein the nucleic acid is a modified ribonucleic acid (RNA).

2. The method of claim 1, wherein, following step (ii), maintaining the target cell under permissible conditions so as to allow the protein of interest to be expressed in the target cell.

3. The method of claim 1, wherein the target cell is present at an anatomic site within a patient's body, wherein step (i) comprises administering to the patient an effective amount of the composition, and wherein step (ii) comprises applying ultrasound to the anatomic site.

4. The method of claim 3, wherein step (i) comprises injection of the composition to the patient.

5. The method of claim 3, wherein the injection is intravenous, intramuscular, subcutaneous, intraperitoneal, or intratumoral injection.

6. The method of claim 3, wherein the patient suffers from a disease characterized by a deficiency of the protein of interest.

7. The method of claim 6, wherein the disease is Fabry Disease and the protein of interest is α-galactosidase (αGAL).

8. The method of claim 3, wherein the anatomic site comprises heart, liver, or kidney.

9. The method of claim 1, wherein the modified RNA comprises the polynucleotide sequence set forth in SEQ ID NO:1.

10. The method of claim 1, wherein the modified RNA comprises 5-methylcytidine and pseudouridine.

11. The method of claim 1, wherein the microbubbles are about 1 μm to about 11 μm in diameter.

12. The method of claim 11, wherein the microbubbles comprise a phospholipid and sulphur hexafluoride.

13. The method of claim 7, wherein the modified RNA is administered to the patient at about 0.5 mg/kg by intravenous injection.

14. The method of claim 13, wherein the modified RNA is administered to the patient once about every 42 days.

15. A composition comprising (1) an effective amount of a modified RNA encoding a protein of interest; (2) microbubbles; and (3) a physiologically acceptable excipient.

16. The composition of claim 15, which is formulated for administration by injection.

17. The composition of claim 15, wherein the modified RNA comprises the polynucleotide sequence set forth in SEQ ID NO:1.

18-20. (canceled)

21. A kit for treating a disease characterized by a deficiency of a protein of interest, comprising (1) a first container containing a first composition comprising microbubbles and an effective amount of a modified RNA encoding the protein of interest; and (2) a second container containing a second composition comprising an effective amount of another therapeutic agent for the disease.

22. The kit of claim 21, wherein the first composition is formulated for administration by injection.

23. The kit of claim 21, wherein the modified RNA comprises the polynucleotide sequence set forth in SEQ ID NO:1.

24-27. (canceled)
